(19) United States
(12) Patent Application Publication
(10) Pub. No.: US 2023/0371869 A1
(43) Pub. Date: Nov. 23, 2023

(54) APPARATUS FOR A MULTIPURPOSE FLUID EXTRACTION DEVICE FOR DIAGNOSTICS AND METHOD OF USE

(71) Applicant: SiPhox, Inc., Burlington, MA (US)

(72) Inventors: Michael Dubrovsky, Somerville, MA (US); Diedrik Rene Vermeulen, Arlington, MA (US)

(73) Assignee: SiPhox, Inc., Burlington, MA (US)

(21) Appl. No.: 18/199,199

(22) Filed: May 18, 2023

Related U.S. Application Data
(60) Provisional application No. 63/343,233, filed on May 18, 2022.

(51) Int. Cl. A61B 5/157; B01L 3/00; A61B 5/1455

(52) U.S. Cl. CPC A61B 5/157; B01L 3/502715; B01L 3/5023; A61B 5/1455; B01L 2300/047; B01L 2200/16; B01L 2300/069; B01L 2300/0636; A61B 2560/0431

(57) ABSTRACT

Apparatus of a multipurpose fluid extraction device for diagnostics and method of use are disclosed. The apparatus includes a fluid extraction system, wherein the fluid extraction system is configured to extract a fluid from a user, a microfluidic assembly, wherein the microfluidic assembly is configured to provide a flow of extracted fluid, where the microfluidic assembly includes a microfluidic channel, a photonic sensor, wherein the photonic sensor is configured to output a sensor signal to a reader device that is configured to detect one or more characteristics of the extracted fluid as a function of the sensor signal, an assay component fluidically connected to the microfluidic assembly, wherein the assay component is configured for testing the extracted fluid and a fluid collecting reservoir fluidically connected to the microfluidic assembly, wherein the fluid collecting reservoir is configured to collect the extracted fluid from the microfluidic assembly.

### CROSS-REFERENCE TO RELATED APPLICATIONS

This application claims the benefit of priority of U.S. Provisional Patent Application Ser. No. 63/343,233 filed on May 18, 2022, and titled “DOUBLE AND TRIPLE PURPOSE BLOOD EXTRACTION DEVICE FOR BLOOD COLLECTION AND ASSAYS USING AN OPTICAL SENSOR AND COLORIMETRIC TEST,” which is incorporated by reference herein in its entirety.

### FIELD OF THE INVENTION

The present invention generally relates to the field of diagnostic devices. In particular, the present invention is directed to apparatus of a multipurpose fluid extraction device for diagnostics and method of use.

### BACKGROUND

A fluid extraction device is a medical device that is used to collect fluid from a patient for diagnostic or therapeutic purposes. Today, fluid extraction devices are widely used in a variety of healthcare settings, including hospitals, clinics, and home healthcare settings. They play a critical role in the diagnosis and management of a wide range of medical conditions. Existing techniques are expensive and time consuming, therefore not sufficient.

### SUMMARY OF THE DISCLOSURE

In an aspect, an apparatus of a multipurpose fluid extraction device for diagnostics is disclosed. The apparatus includes a fluid extraction system, wherein the fluid extraction system is configured to extract a fluid from a user, a microfluidic assembly, wherein the microfluidic assembly is configured to provide a flow of extracted fluid, where the microfluidic assembly includes a microfluidic channel, a photonic sensor, wherein the photonic sensor is configured to output a sensor signal to a reader device, a reader device, wherein the reader device is configured to detect one or more characteristics of the extracted fluid as a function of the sensor signal, an assay component fluidically connected to the microfluidic assembly, wherein the assay component is configured for testing the extracted fluid and a fluid collecting reservoir fluidically connected to the microfluidic assembly, wherein the fluid collecting reservoir is configured to collect the extracted fluid from the microfluidic assembly.

In another aspect, a method of use of a multipurpose fluid extraction device for diagnostics is disclosed. The method includes extracting, using a fluid extraction system, a fluid from a user, providing, using a microfluidic assembly, a flow of extracted fluid, wherein the microfluidic assembly comprises a microfluidic channel, outputting, using a photonic sensor, a sensor signal to a reader device, detecting, using a reader device, one or more characteristics of the extracted fluid as a function of the sensor signal, testing, using an assay component fluidically connected to the microfluidic assembly, the extracted fluid and collecting, using a fluid collecting reservoir fluidically connected to the microfluidic assembly, the extracted fluid from the microfluidic assembly.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1A is an illustration of an exemplary embodiment of an exploded view of an apparatus of a multipurpose fluid extraction device for diagnostics;

FIG. 1B is an illustration of an exemplary embodiment of an assembled view of an apparatus of a multipurpose fluid extraction device for diagnostics;

FIG. 2 is a block diagram of an exemplary workflow of an apparatus of a multipurpose fluid extraction device for diagnostics;

FIGS. 3A-C are illustrations of exemplary embodiments of a fluid extraction device and its use;

FIGS. 4A-D are illustrations of cross section views of exemplary embodiments of a microfluidic assembly;

FIGS. 5A-B are illustrations of exemplary embodiments of a portion of an apparatus with an assay component and a fluid collecting reservoir removably connected to a housing;

FIGS. 6A-B are illustrations of exemplary embodiments of a portion of an apparatus with an assay component and a photonic sensor;

FIG. 7 is an illustration of an exemplary embodiment of a portion of an apparatus with an assay component and a test timing feature;

FIG. 8 is a flow diagram of an exemplary method of use of multipurpose fluid extraction device for diagnostics; and

FIG. 9 is a block diagram of a computing system that can be used to implement any one or more of the methodologies disclosed herein and any one or more portions thereof.

The drawings are not necessarily to scale and may be illustrated by phantom lines, diagrammatic representations, and fragmentary views. In certain instances, details that are not necessary for an understanding of the embodiments or that render other details difficult to perceive may have been omitted.

### DETAILED DESCRIPTION

At a high level, apparatus of a multipurpose fluid extraction device for diagnostics and method of use are disclosed. The apparatus includes a fluid extraction system, wherein the fluid extraction system is configured to extract a fluid from a user, a microfluidic assembly, wherein the microfluidic assembly is configured to provide a flow of extracted fluid, where the microfluidic assembly includes a microfluidic channel, a photonic sensor, wherein the photonic sensor is configured to output a sensor signal to a reader device, a reader device, wherein the reader device is configured to detect one or more characteristics of the extracted fluid as a function of the sensor signal, an assay component fluidically connected to the microfluidic assembly, wherein the assay component is configured for testing the extracted fluid and a fluid collecting reservoir fluidically connected to the microfluidic assembly, wherein the fluid collecting reservoir is configured to collect the extracted fluid from the microfluidic assembly.

Aspects of the present disclosure allow for fluid collection for future testing and fluid assays tests over a photonics sensor that enables digital capture and analysis. In some embodiments, a patient can collect blood for further analysis and getting an immediate initial measurement. In some embodiments, the present disclosure can extract, collect, and test blood in a single application and give the patient the flexibility of in-mail testing but also an immediate test result.

The present disclosure may also discuss a photonic biosensor that could provide an inexpensive, re-usable, and scalable diagnostic solution for sensing of an array of biological analytes with enhanced specificity and sensitivity of detection. Exemplary embodiments illustrating aspects of the present disclosure are described below in the context of several specific examples.

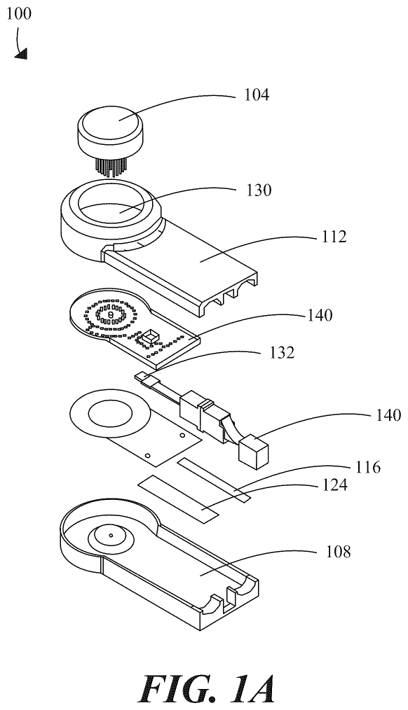

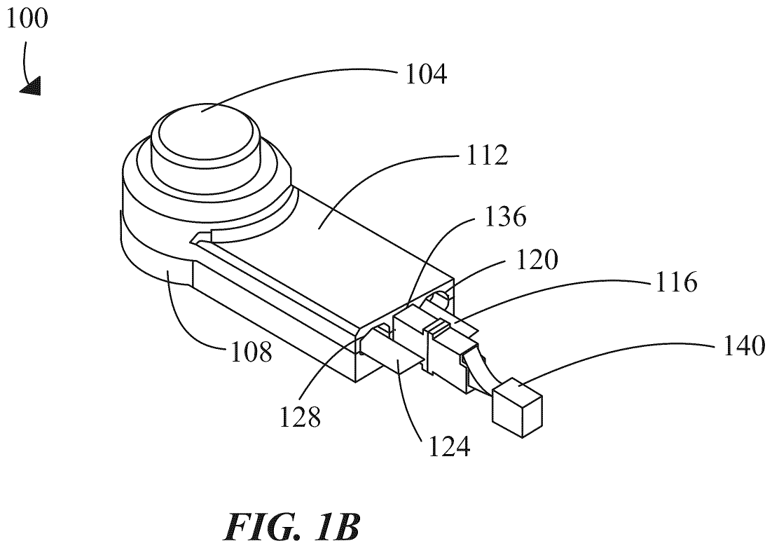

Referring now to FIGS. 1A-B, FIG. 1A illustrates an exemplary embodiment of an exploded view of an apparatus 100 of a fluid extraction device 104 for diagnostics. FIG. 1B illustrates an exemplary embodiment of an assembled view of the apparatus 100 of the fluid extraction device 104 for diagnostics. In some embodiments, the apparatus 100 may be reusable. In some embodiments, the apparatus 100 may include a housing. As used in this disclosure, a “housing” refers to an outer structure configured to contain a plurality of components. In some embodiments, this may include, without limitation, components of apparatus 100 as described in this disclosure. In some embodiments, the housing may be portable. For the purposes of this disclosure, a “portable” refers to the capability for an object to be easily carried or moved from place to place. In some cases, the housing may include a durable, lightweight material such as without limitation, plastic, metal, and/or the like. In some embodiments, the housing may be designed and configured to protect sensitive components of apparatus 100 from damage or contamination. In some embodiments, the housing may include one or more physical notches and/or grooves that allow for precise placement of devices and/or components. In some embodiments, the housing may include one or more optical markers or alignment indicators that are visible (through human eye, microscope, any other imaging system, and/or the like) and allow for accurate positioning of devices and/or components. In some embodiments, the housing may include one or more surface coatings and/or modifications that reduce the likelihood of unwanted adhesion or interference with external components and/or substances. Additionally, or alternatively, the housing may further include features such as latches, clips, or other fasteners that help to secure apparatus 100 in place during use.

With continued reference to FIGS. 1A-B, in some embodiments, a housing may include a first housing 108 and a second housing 112 as shown in FIGS. 1A-B. As a non-limiting example, the second housing 112 may be placed on atop of the first housing 108. The first housing 108 and the second housing 112 may allow the housing to be assembled or disassembled easily. In an embodiment, the first housing 108 and the second housing 112 may be matched permanently. As a non-limiting example, the first housing and the second housing 112 may be permanently connected to each other using a variety of techniques, such as but not limited to welding, soldering, brazing, adhesive bonding, or mechanical fasteners. In another embodiment, the first housing 108 and the second housing 112 may be matched temporarily. In another word, the first housing 108 and the second housing 112 may be removably connected to each other. As a non-limiting example, the first housing 108 and the second housing 112 may be removably connected to each other using a mechanical fasteners may include bolts, screws, nuts, washers, rivets, pins, and the like. For the purposes of this disclosure, “removably connected” refers to an ability for an object that is connected to another object to be disconnected from the other object without damaging or breaking said objects. In some embodiments, the removable connection may include threaded connection. For the purposes of this disclosure, “threaded connection” is a type of connection that involves mating male and female halves together to create a connection to hold the threads together. As a non-limiting example, the threaded connection may be done by way of gendered mating components. As a non-limiting example, the gendered mating components may include a male component or plug which is inserted within a female component or socket. In some cases, the threaded connection may be removable. In some cases, the threaded connection may be removable, but requires a specialized tool or key for removal. In some embodiments, the threaded connection may be achieved by way of one or more of plug and socket mates, pogo pin contact, crown spring mates, and the like. In some cases, the threaded connection may be keyed to ensure proper alignment of a mating component. In some cases, the threaded connection may be lockable. As used in this disclosure, a “mating component” is a component that mates with at least another component. As a non-limiting example, the mating component may include a mechanical connector. In another embodiment, the removable connection may include bayonet connections. The bayonet connections use a locking mechanism that allows the two components to be connected by inserting and twisting them into place. In another embodiment, the removable connection may include snap-fit connections. In some embodiments, the snap-fit connections may include a series of tabs or hooks that snap into place when the two components are pushed together. As a non-limiting example, the snap-fit connections may include snap-fit clips, snap-fit tabs, snap-fit hinges, snap-fit latches, snap-fit hooks, snap-fit pins, and the like. In another embodiment, the removable connection may include latch connections. The latch connections use a latch or locking mechanism that secures the two components together. As a non-limiting example, the latch connections may include cabinet latches, door latches, aircraft fasteners, and the like. In another embodiment, the removable connection may include clamp connections. In some embodiments, the clamp connections use a clamp or compression mechanism to hold the two components together. As a non-limiting example, the clamp connections may include hose clamps, c-clamps, pipe clamps, wire rope clamps, shaft collars, spring clamps, and the like. In another embodiment, the removable connection may include magnetic connections. In some embodiments, the magnetic connections use magnets to hold the two components together. In some embodiments, the removable connection may include connectors, screws, adapters, feedthrough, and the like. For the purposes of this disclosure, a “connector” is a component configured to create an electrical or mechanical connection between two or more objects. Examples of connectors include plug and socket connectors, terminal blocks, crimp connectors, and the like.

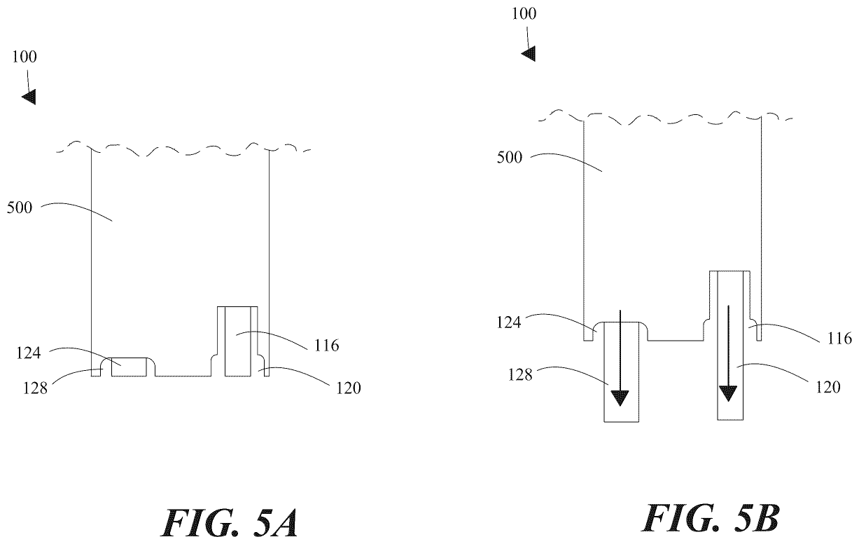

With continued reference to FIGS. 1A-B, in some embodiments, a housing may include at least an aperture that provides a path for a connection between components of an apparatus 100 for communication. For the purposes of this disclosure, an “aperture” is an opening or hole through which something passes or can be seen. As a non-limiting example, an assay component 116 may be removably inserted in the housing using a first aperture 120 of the housing as shown in FIGS. 5A-B. For example, and without limitation, the assay component 116 may be removably inserted into the housing through the first aperture 120 for testing at least a fluid and removed once the testing is finished. As another non-limiting example, a fluid collecting reservoir 124 may be removably inserted in the housing using a second aperture 128 as shown in FIGS. 5A-B. For example, and without limitation, the fluid collecting reservoir 124 may be removably inserted into the housing through the second aperture 128 for collecting the at least a fluid and removed through the second aperture 128 once the at least a fluid is collected. For the purposes of this disclosure, “removably inserted” refers to an object that has been inserted or placed an into another object such that the object can be removed from the other object without causing damage or leaving any residue behind. As another non-limiting example, a fluid extraction system 104 may be removably connected to the housing using a third aperture 130 of the housing. For example, and without limitation, the fluid extraction system 104 may be removably connected to the third aperture 130 of the housing using a snap-fit connections as described above. The assay component 116, the fluid collecting reservoir 124 and the fluid extraction system 104 disclosed herein are described further in detail below. As another non-limiting example, a photonic sensor 132 may be removably inserted into the housing using a fourth aperture 136 of the housing 500 as shown in FIG. 1B. As another non-limiting example, the photonic sensor 132 may be removably connected into the housing using the fourth aperture 136 of the housing using a snap-fit connection as described above. As another non-limiting example, the photonic sensor 132 may be removed from the housing when a first housing 108 and a second housing 112 get disassembled.

With continued reference to FIGS. 1A-B, in some embodiments, a housing may include one or more bumps inside the housing that allow for precise placement of devices and/or components. As a non-limiting example, one or more bumps may be present to indicate the extent to which a fluid collecting reservoir 124 and/or an assay component 116 can be removably inserted into the housing through at least an aperture, such as a first aperture 120 and a second aperture 128. When, for example, and without limitation, the fluid collecting reservoir 124 and/or assay component 116 is being inserted into the housing through at least one aperture, the one or more bumps may prevent further placement into the housing. In some embodiments, the housing may include one or more optical markers or alignment indicators that are visible (through human eye, microscope, any other imaging system, and/or the like) and allow for accurate positioning of devices and/or components, such as but not limited to the fluid collecting reservoir 124, the assay component 116 and/or a fluid extraction system 104. In some embodiments, the fluid collecting reservoir 124, the assay component 116 and/or the fluid extraction system 104 may include the one or more optical markers or alignment indicators that are visible (through human eye, microscope, any other imaging system, and/or the like) and allow for accurate positioning of them into the housing.

With continued reference to FIGS. 1A-B, as used in this disclosure, “communication” is an attribute wherein two or more relata interact with one another, for example within a specific domain or in a certain manner. In some cases, communication between two or more relata may be of a specific domain, such as without limitation electric communication, fluidic communication, informatic communication, mechanical communication, and the like. As used in this disclosure, “informatic communication” is an attribute wherein two or more relata interact with one another by way of an information flow or information in general. For example, and without limitation, a communication between a photonic sensor 132 and a reader device 140 may include the informatic communication. The photonic sensor 132 and the reader device 140 disclosed herein are described in detail below. As used in this disclosure, “mechanic communication” is an attribute wherein two or more relata interact with one another by way of mechanical means, for instance mechanical effort (e.g., force) and flow (e.g., velocity). “Electric communication,” as used in this disclosure, is an attribute wherein two or more relata interact with one another by way of an electric current or electricity in general. “Fluidic communication,” as used in this disclosure, is an attribute wherein two or more relata interact with one another by way of a fluidic flow or fluid in general. For example, and without limitation, a communication between a microfluidic assembly 140 and a fluid collecting reservoir 124 may include the fluidic communication. For example, and without limitation, a communication between the microfluidic assembly 140 and an assay component 116 may include the fluidic communication. For example, and without limitation, a communication between the microfluidic assembly 140 and a fluid extraction system 104 may include the fluidic communication.

With continued reference to FIGS. 1A-B, an apparatus 100 includes a fluid extraction system 104. For the purposes of this disclosure, a “fluid extraction system” is a system that is configured to extract a fluid from a user. For the purposes of this disclosure, a “user” is any human or animal. In some embodiments, the “user” may be consistent with a “patient” In some embodiments, the fluid extraction system 104 may be disposable after use. In some embodiments, the fluid extraction system 104 may be replaceable. For the purposes of this disclosure, “fluid” is any sample that has no fixed shape and yields easily to external pressure. For the purposes of this disclosure, a “sample” is some quantity of tissue, fluid, or the like extracted from a subject organism, such as without limitation, a human being, and/or a substance derived therefrom. As a non-limiting example, the fluid may include cerebrospinal fluid, whole blood, urine samples, and the like. In some embodiments, the fluid may include one or more analytes. For the purposes of this disclosure, an “analyte” is a substance that is of interest in an analytical procedure. As a non-limiting example, the one or more analytes may include glucose, proteins, hormones, antibodies, and the like. As another non-limiting example, the one or more analytes may include Albumin, C-reactive protein, SARS-COV-2 protein, Thyroxine-binding globulin, Thyroxine-binding prealbumin (transthyretin), Ceruloplasmin, Haptoglobin, Apolipoprotein A-I (protein of HDL), Apolipoprotein A-II (another protein of HDL), Apolipoprotein B-100 (protein of LDL), Transferrin, Serum free light chains (info from LabCorp), Antithrombin III, Fibrinogen, Lysozyme, Plasminogen, C3 complement, C4 complement, D-dimer, a1-Fetoprotein (AFP), a2-Macroglobulin (AMG), Retinol binding protein, Alpha1-Antitrypsin (A1AT or AAT), a1-Acid Glycoprotein (or orosomucoid), cxl-antichymotrypsin (Serpin family A member 3) ghrelin, Hemopexin, Complement factor H, Vitronectin, C4b binding protein (Complement component 4 binding protein beta), Cysteine rich secretory glycoprotein LCCL domain containing 2 (Crispld2), Complement C5, Alpha 1-B glycoprotein, Apolipoprotein H, Apolipoprotein A4, Plasminogen, GC vitamin D binding protein (DBP), Histidine rich glycoprotein, Coagulation factor II, thrombin, Glycosylphosphatidylinositol specific phospholipase Dl, Complement Cls, Fetuin B, Kininogen 1, Complement C9, Gelsolin, Apolipoprotein C3, Serpin family A member 6, Apolipoprotein C1, Paraoxonase 1, Serum amyloid 4, Alpha-2 glycoprotein 1, zinc-binding, Afamin, Apolipoprotein C2, Clusterin, Apolipoprotein E, Serpin family A member 7, Complement component 4 binding protein alpha, Kallikrein B1, Amyloid P component, Renalase, FAD dependent amine oxidase, Thrombospondin 1, Leucine rich alpha-2 glycoprotein 1, Lipopolysaccharide binding protein, Protein S, Retinal binding protein 4, Apolipoprotein F, Ficolin 3, Phospholipase transfer protein, Serpin family F member 1, Adiponectin, C1Q and collagen domain, Insulin such as growth factor binding acid labile subunit, Ficolin 2, Hyaluronan binding protein 2, Mannan binding lectin serine peptidase 1, C-type lectin domain family 3 member B, Coagulation factor V, Complement Clr subcomponent, Lecithin-cholesterol acyltransferase, CDS molecule, Serpin family A member 10, Apolipoprotein L1, Insulin like growth factor binding protein 3, Cholesterol ester transfer protein, CD14, Glutathione peroxidase 3, CD163, Paraoxanase 3, Protein Z, Ficolin 1, Transferrin receptor, ADAM metallopeptidase with thrombospondin type 1 motif 13, Complement factor D, Cystatin C, Apolipoprotein C4, Myeloperoxidase, Mannose binding lectin 2, Complement factor B, C-C motif chemokine ligand 28, Tenascin C, Vascular cell adhesion molecule 1 (VCAM1), Cathelicidin antimicrobial peptide, Insulin like growth factor binding protein 2, Complement factor H related 3, Insulin like growth factor 2, Complement Clq C chain, Mannan binding lectin serine peptidase 2, Lipase G, C1q and TNF related 9, Fibrinogen alpha chain, Clq and TNF related 6, Von Willebrand factor, Gremlin 1, C1q and TNF related 5, C1q and TNF related 1, Serum amyloid A1, Angiogenin, C1q and TNF related 7, Orosomucoid 2, Angiopoietin like 3, Fc receptor like BMP4, Chromogranin A, and the like. Persons skilled in the art, upon reviewing the entirety of this disclosure, may appreciate various analytes that may be used for an apparatus 100. Additional disclosures related to the at least an analyte may be found in International Patent Application No PCT/US2022/037767, filed on Jul. 20, 2022, entitled as “WEARABLE BIOSENSORS FOR SEMI-INVASIVE, REAL-TIME MONITORING OF ANALYTES, AND RELATED METHODS AND APPARATUS,” the entirety of which is incorporated herein by reference.

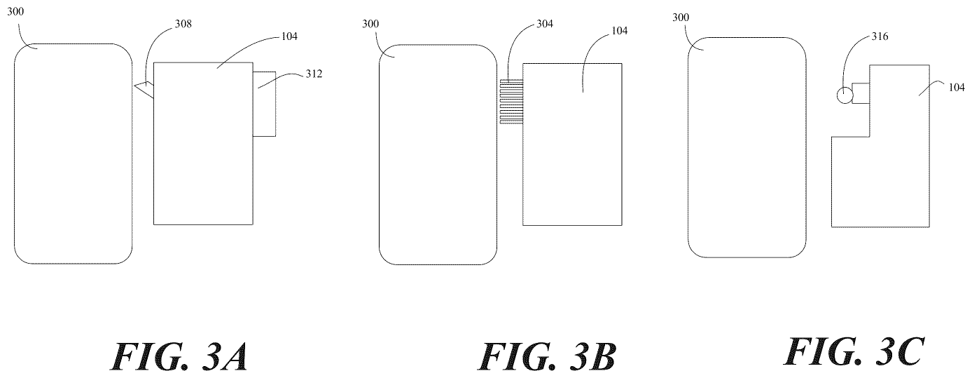

With continued reference to the FIGS. 1A-B, in some embodiments, a fluid extraction system 104 may include an optical skin piercing component. For the purposes of this disclosure, an “optical skin piercing component” is a device that uses light-based technology to pierce the skin. In some embodiments, the optical skin piercing component may include a light source to create a beam of light that can be focused on a specific area of the skin to create a hole or puncture. In some embodiments, the light source may produce a narrow, focused beam of light that can be precisely controlled in terms of wavelength, pulse duration, size and shape of the beam, and intensity. As a non-limiting example, the optical skin piercing component may include a laser lancet as shown in FIG. 3C. In some embodiments, the laser lancets may emit a focused beam of light that heats the skin at a specific location, causing it to vaporize and create a small incision. In some embodiments, the laser beam may be controlled by a computing device. The computing device disclosed herein may be consistent with any computing device disclosed in the entirety of this disclosure. As a non-limiting example, the computing device may control over the size and depth of the incision. In some embodiments, the optical skin piercing component may be actuated using an actuator described below. As a non-limiting example, a user may push a button to actuate the optical skin piercing component.

With continued reference to the FIGS. 1A-B, in another embodiment, the fluid extraction system 104 may include a mechanical skin piercing component. As a non-limiting example, the mechanical skin piercing component may include microneedles, micro blades, and the like as shown in FIGS. 3A-B. As another non-limiting example, the mechanical skin piercing component may include a lancet. In some embodiments, the mechanical skin piercing component may include stainless steel or other metals. In some embodiments, the mechanical skin piercing component may include various sizes and shapes. In an embodiment, the mechanical skin piercing component may be manually operated. As a non-limiting example, a user may manually make an incision on the skin using the mechanical skin piercing component. As another non-limiting example, the user may manually make an incision on the skin by pushing an actuator, such as but not limited to a button, to actuate the mechanical skin piercing component. In another embodiment, the mechanical skin piercing component may be automated. As a non-limiting example, the mechanical skin piercing component may be automated using a computing device. For example, and without limitation, the computing device may be implemented in a reader device 140. In an embodiment, the mechanical skin piercing component can be used for single or multiple punctures.

With continued reference to the FIGS. 1A-B, in some embodiments, the fluid extraction system 104 may include an actuator. For the purposes of this disclosure, an “actuator” is a component of a device that is responsible for moving and/or controlling a mechanism or system. As a non-limiting example, the actuator may include a button, lever, and the like. In some embodiments, the actuator may be pressed or clicked to perform a specific action. As a non-limiting example, the actuator may be configured to actuate the optical skin piercing component of a fluid extraction system 104. As another non-limiting example, the actuator may be configured to actuate a mechanical skin piercing component of the fluid extraction system 104. In some embodiments, the actuator may be controlled by a computing device.

With continued reference to FIGS. 1A-B, an actuator may include a component of a machine that is responsible for moving and/or controlling a mechanism or system. An actuator may, in some cases, require a control signal and/or a source of energy or power. In some cases, a control signal may be relatively low energy. Exemplary control signal forms include electric potential or current, pneumatic pressure or flow, or hydraulic fluid pressure or flow, mechanical force/torque or velocity, or even human power. In some cases, an actuator may have an energy or power source other than control signal. This may include a main energy source, which may include for example electric power, hydraulic power, pneumatic power, mechanical power, and the like. In some cases, upon receiving a control signal, an actuator responds by converting source power into mechanical motion. In some cases, an actuator may be understood as a form of automation or automatic control.

With continued reference to FIGS. 1A-B, in some embodiments, actuator may include a hydraulic actuator. A hydraulic actuator may consist of a cylinder or fluid motor that uses hydraulic power to facilitate mechanical operation. Output of hydraulic actuator may include mechanical motion, such as without limitation linear, rotatory, or oscillatory motion. In some cases, hydraulic actuator may employ a liquid hydraulic fluid. As liquids, in some cases. are incompressible, a hydraulic actuator can exert large forces. Additionally, as force is equal to pressure multiplied by area, hydraulic actuators may act as force transformers with changes in area (e.g., cross sectional area of cylinder and/or piston). An exemplary hydraulic cylinder may consist of a hollow cylindrical tube within which a piston can slide. In some cases, a hydraulic cylinder may be considered single acting. Single acting may be used when fluid pressure is applied substantially to just one side of a piston. Consequently, a single acting piston can move in only one direction. In some cases, a spring may be used to give a single acting piston a return stroke. In some cases, a hydraulic cylinder may be double acting. Double acting may be used when pressure is applied substantially on each side of a piston; any difference in resultant force between the two sides of the piston causes the piston to move.

With continued reference to FIGS. 1A-B, in some embodiments, actuator may include a pneumatic actuator. In some cases, a pneumatic actuator may enable considerable forces to be produced from relatively small changes in gas pressure. In some cases, a pneumatic actuator may respond more quickly than other types of actuators, for example hydraulic actuators. A pneumatic actuator may use compressible fluid (e.g., air). In some cases, a pneumatic actuator may operate on compressed air. Operation of hydraulic and/or pneumatic actuators may include control of one or more valves, circuits, fluid pumps, and/or fluid manifolds.

With continued reference to FIGS. 1A-B, in some cases, actuator may include an electric actuator. Electric actuator may include any electromechanical actuators, linear motors, and the like. In some cases, actuator may include an electromechanical actuator. An electromechanical actuator may convert a rotational force of an electric rotary motor into a linear movement to generate a linear movement through a mechanism. Exemplary mechanisms, include rotational to translational motion transformers, such as without limitation a belt, a screw, a crank, a cam, a linkage, a scotch yoke, and the like. In some cases, control of an electromechanical actuator may include control of electric motor, for instance a control signal may control one or more electric motor parameters to control electromechanical actuator. Exemplary non-limitation electric motor parameters include rotational position, input torque, velocity, current, and potential. electric actuator may include a linear motor. Linear motors may differ from electromechanical actuators, as power from linear motors is output directly as translational motion, rather than output as rotational motion and converted to translational motion. In some cases, a linear motor may cause lower friction losses than other devices. Linear motors may be further specified into at least 3 different categories, including flat linear motor, U-channel linear motors and tubular linear motors. Linear motors may be directly controlled by a control signal for controlling one or more linear motor parameters. Exemplary linear motor parameters include without limitation position, force, velocity, potential, and current.

With continued reference to FIGS. 1A-B, in some embodiments, an actuator may include a mechanical actuator. In some cases, a mechanical actuator may function to execute movement by converting one kind of motion, such as rotary motion, into another kind, such as linear motion. An exemplary mechanical actuator includes a rack and pinion. In some cases, a mechanical power source, such as a power take off may serve as power source for a mechanical actuator. Mechanical actuators may employ any number of mechanism, including for example without limitation gears, rails, pulleys, cables, linkages, and the like.

With continued reference to the FIGS. 1A-B, an apparatus 100 includes a microfluidic assembly 140. For the purposes of this disclosure, a “microfluidic assembly” is an assembly that is configured to act upon fluids at a small scale, such as without limitation a sub-millimeter scale. At small scales, surface forces may dominate volumetric forces. In some embodiments, the microfluidic assembly 140 is configured to provide a flow of extracted fluid. In some embodiments, plasma may be separated in the microfluidic assembly 140. For the purposes of this disclosure, “extracted fluid” is a fluid that is extracted from the skin using a fluid extraction system. In some embodiments, the microfluidic assembly 140 may be configured to provide the flow of the extracted fluid over a photonic sensor 132. As a non-limiting example, the microfluidic assembly 140 may be on atop one or more resonators of the photonic sensor 132. The photonic sensor 132 and the one or more resonators disclosed herein are further described below. Additional disclosure related to the microfluidic assembly 140 may be found in U.S. patent application Ser. No. 18/121,712, filed on Mar. 15, 2023, entitled “APPARATUS AND METHODS FOR PERFORMING MICROFLUIDIC-BASED BIOCHEMICAL ASSAYS,” and in U.S. patent application Ser. No. 17/859,932, filed on Jul. 7, 2022, entitled “SYSTEM AND METHODS FOR FLUID SENSING USING PASSIVE FLOW,” the entirety of which are incorporated herein by references.

With continued reference to FIGS. 1A-B, in some embodiments, a microfluidic assembly 140 includes a microfluidic channel. As used in this disclosure, a “microfluidic channel” is a structure within microfluidic assembly that is designed and/or configured to manipulate one or more fluids at micro scale. In some cases, microfluidic channel may enable a precise manipulation of fluids and samples in a controlled and/or reproducible manner within microfluidic assembly 140. In some embodiments, microfluidic channel of microfluidic assembly 140 may be designed and arranged based on particular needs. In other embodiments, the microfluidic channel of microfluidic assembly 140 may be varied depending on the type of a fluid being used, that is directly contact with microfluidic channel. In a non-limiting example, attributes of the microfluidic channel such as, without the size and/or shape of the substrate may be determined as a function of specific assay protocols.

With continued reference to FIG. 1, in some embodiments, a microfluidic assembly 140 may further include at least a flow component connected with at least a microfluidic channel configured to flow at least a fluid through a photonic sensor 132. In some embodiments, at least a flow component may include a passive flow component configured to initiate a passive flow process. In some embodiments, the passive flow component may be in fluidic communication with a fluid collecting reservoir 124. The fluid collecting reservoir 124 disclosed herein is further described below. As a non-limiting example, the fluid collecting reservoir 124 may be configured to passively pump the extracted fluid for the flow of the extracted fluid of the microfluidic assembly 140. For the purposes of this disclosure, “passively pump” refers to pumping a fluid without an external actuator or power source. For example, and without limitation, the microfluidic assembly 140 and the fluid collecting reservoir 124 may create a concentration gradient that drives the flow of fluids through the microfluidic assembly 140 (e.g. osmosis). In some embodiments, the passive flow component may be in fluidic communication with an assay component 116. As used in this disclosure, a “passive flow component” is a component, typically of a microfluidic assembly, that imparts a passive flow on at least a fluid, wherein the “passive flow,” for the purpose of this disclosure, is flow of the at least a fluid, which is induced absent any external actuators, fields, or power sources. As used in this disclosure, a “passive flow process” is a plurality of actions or steps taken on passive flow component in order to impart a passive flow on at least a fluid. In some embodiments, the passive flow component may employ one or more passive flow techniques in order to initiate passive flow process; for instance, and without limitation, passive flow techniques may include osmosis, capillary action, surface tension, pressure, gravity-driven flow, hydrostatic flow, vacuums, and the like. As a non-limiting example, the capillary action can occur when a fluid flows through a microfluidic channel due to the adhesive and cohesive properties of the fluid. When the fluid encounters a surface, such as the walls of the microfluidic channel, the fluid can be drawn into the microfluidic channel by the capillary action. The fluid can then be transported through the microfluidic channel by the combined forces of adhesion and cohesion, which cause the fluid to flow along the microfluidic channel's surface. As another non-limiting example, the gravity-driven flow may allow the fluid to flow downhill due to the force of gravity. In a non-limiting example, the passive flow component may be consistent with any passive flow component described in U.S. patent application Ser. No. 18/121,712, filed on Mar. 15, 2023, entitled “APPARATUS AND METHODS FOR PERFORMING MICROFLUIDIC-BASED BIOCHEMICAL ASSAYS,” and in U.S. patent application Ser. No. 17/859,932, filed on Jul. 7, 2022, entitled “SYSTEM AND METHODS FOR FLUID SENSING USING PASSIVE FLOW,” the entirety of which are incorporated herein by references.

With continued reference to FIGS. 1A-B, in other embodiments, at least a flow component may include an active flow component configured to initiate an active flow process. As used in this disclosure, an “active flow component” is a component that imparts an active flow on at least a fluid, wherein the “active flow,” for the purpose of this disclosure, is flow of the at least a fluid which is induced by external actuators, fields, or power sources. As used in this disclosure, an “active flow process” is a plurality of actions or steps taken on active flow component in order to impart active flow on at least a fluid. In some embodiments, the active flow component is in fluidic communication with a fluid collecting reservoir 124. In some embodiments, the active flow component may be in fluidic communication with an assay component 116. In a non-limiting example, the active flow component may include one or more pumps. The one or more pumps may include a substantially constant pressure pump (e.g., centrifugal pump) or a substantially constant flow pump (e.g., positive displacement pump, gear pump, and the like). The one or more pumps can be hydrostatic or hydrodynamic. As used in this disclosure, a “pump” is a mechanical source of power that converts mechanical power into fluidic energy. The one or more pumps may generate flow with enough power to overcome pressure induced by a load at a pump outlet. The one or more pumps may generate a vacuum at a pump inlet, thereby forcing fluid into the pump inlet to the one or more pumps pump and by mechanical action delivering the fluid to a pump outlet. The hydrostatic pumps may include positive displacement pumps. The hydrodynamic pumps can be fixed displacement pumps, in which displacement may not be adjusted, or variable displacement pumps, in which the displacement may be adjusted. Exemplary non-limiting pumps include gear pumps, rotary vane pumps, screw pumps, bent axis pumps, inline axial piston pumps, radial piston pumps, and the like. The one or more pumps may be powered by any rotational mechanical work source, for example without limitation and electric motor or a power take off from an engine. The one or more pumps may be in fluidic communication with the fluid collecting reservoir 124.

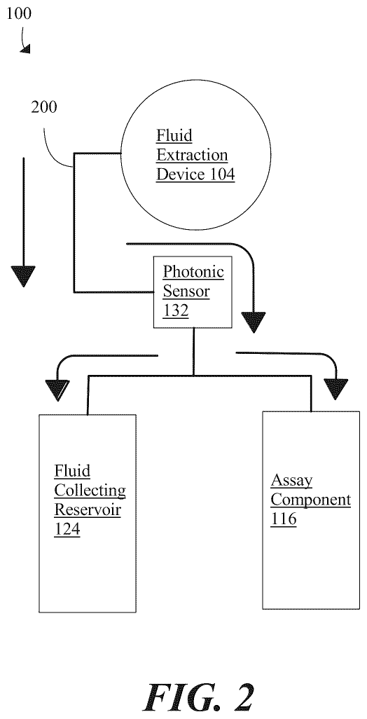

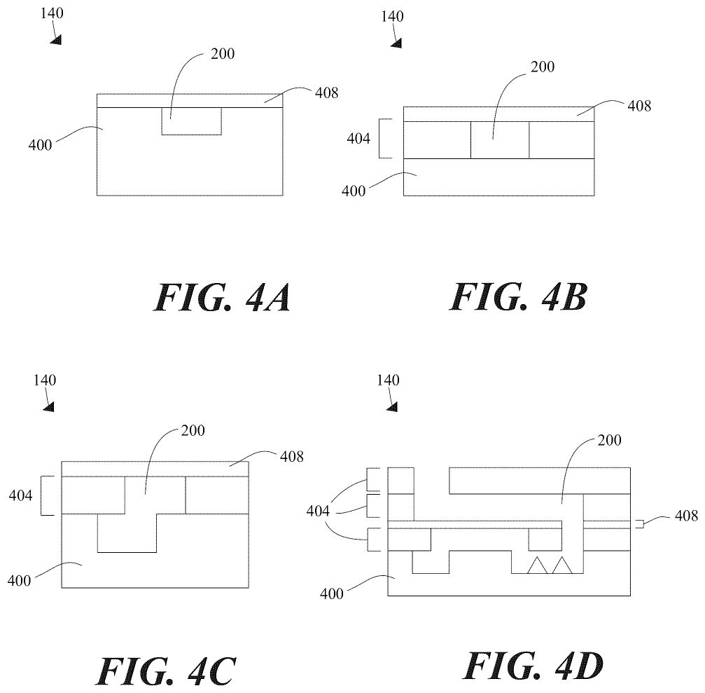

With continued reference to FIGS. 1A-B, in some embodiments, a microfluidic assembly 140 may include a multiple layers of a microfluidic channel as shown in FIGS. 4B-D. As a non-limiting example, the multiple layers of the microfluidic channel may be created by multilayering Pressure Sensitive adhesive (PSA) over etched elements on the piece or other PSA laminates. In some embodiments, the microfluidic channel of the microfluidic assembly 140 may be sealed with adhesive laminate. In some embodiments, the microfluidic channel of the microfluidic assembly may be formed by various techniques such as but not limited to photolithography, etching, deposition, and/or other microfabrication techniques. As a non-limiting example, the microfluidic channel may be etched on a substrate such as but not limited to glass, silicon, or polymers. In some embodiments, the microfluidic channels of the microfluidic assembly 140 may include various size depending on capillary needs as well as characteristics of fluids. In some embodiments, the microfluidic assembly may be configured to drive the fluid over a photonic sensor 132 and then to an assay component 116 and/or a fluid collecting reservoir 124. An exemplary configuration of the flow of the fluid is illustrated in FIG. 2.

With continued reference to FIGS. 1A-B, in some embodiments, a microfluidic assembly may include an amplifier. As used in this disclosure, an “amplifier” is a component that can increase the sensitivity of the system to detect specific analytes. For an amplified binding, the amplifiers can be placed on microfluidic channels to later be driven alongside the extracted fluid onto a photonic sensor 132. In some embodiments, the amplifier may amplify signals generated by the binding of a target analyte to a probe molecule, such as but not limited to a binding ligand as described above, which may allow for more precise and accurate detection of the target analyte. In an embodiment, the amplifier may include a biochemical amplifier. For example, and without limitation, the biochemical amplifier may include a nanoparticle such as but not limited to magnetic beads nanoparticles, gold nanoparticles, magnetic nanoparticle, magnetic gold particle, and other magnetic detection particles. As another non-limiting example, the amplifier may include an enzyme, such as but not limited to horseradish peroxidase (HRP) or alkaline phosphatase (ALP). As another non-limiting example, the amplifier may include nucleic acid amplification such as but not limited to Polymerase chain reaction (PCR). In another embodiment, the amplifier may include an electromechanical amplifier. For example, and without limitation, the electromechanical amplifier may include cyclic voltammetry or chronoamperometry. Additional disclosure related to the amplifier may be found in U.S. patent application Ser. No. 18/199,171, filed on May 18, 2023, entitled “APPARATUS AND METHOD FOR DETECTING AN ANALYTE,” having an attorney docket number of 1214-013USU1, the entirety of which is incorporated herein as a reference.

With continued reference to FIGS. 1A-B, an apparatus 100 includes a photonic sensor 132. For the purposes of this disclosure, “photonic sensor” a sensor that includes electronic components that form a functional circuit that detects light. In some embodiments, the photonic sensor 132 is configured to output a sensor signal to a reader device 140 as described below. Additionally and without limitation, the photonic sensor 132 disclosed herein may be consistent with a sensor device found in U.S. patent application Ser. No. 18/121,712, filed on Mar. 15, 2023, entitled as “APPARATUS AND METHODS FOR PERFORMING MICROFLUIDIC-BASED BIOCHEMICAL ASSAYS,” having an attorney docket number of 1214-008USU1, and a photonic sensor chip in U.S. patent application Ser. No. 18/126,014, filed on Mar. 24, 2023, entitled as “PHOTONIC BIOSENSORS FOR MULTIPLEXED DIAGNOSTICS AND A METHOD OF USE,” having an attorney docket number of 1214-010USU1, the entirety of which are incorporated herein by reference.

With continued reference to FIGS. 1A-B, in some embodiments, a photonic sensor 132 may include an optical waveguide. For the purposes of this disclosure, an “optical waveguide” is a structure that is designed to confine and guide electromagnetic waves along a path from one point to another. As a non-limiting example, electromagnetic waves may include ultraviolet, x-rays, gamma rays, infrared, microwave, radio waves, visible light, and the like. In some embodiments, the photonic sensor 132 may include a plurality of optical waveguide. In some embodiments, the optical waveguide may include dielectric materials, silicon, glass, polymer, semiconductor, and the like. In some embodiments, the optical waveguide may include various geometry of the waveguide. As a non-limiting example, the optical waveguide may include a straight waveguide, tapered waveguide, grating waveguide, and the like. In some embodiments, the optical waveguide may include various shapes, such as but not limited to rectangular, circular, elliptical cross-sections, and the like. In some embodiments, the optical waveguide may include optical fiber waveguides, transparent dielectric waveguides, liquid light guides, liquid waveguides, light pipe, laser-inscribed waveguide, and the like. In some embodiments, the optical waveguide may include planar, strip, rib, fiber waveguides, and the like. In some embodiments, the optical waveguide may include single-mode, multi-mode, and the like. In some embodiments, the optical waveguide may include various refractive index distributions such as but not limited to step index distribution, gradient index distribution, and the like. For the purposes of this disclosure, “refractive index” of a material is a measure of how much the material can bend, or refract, light as it passes through it.

With continued reference to FIGS. 1A-B, in some embodiments, an optical waveguide may be configured to output an optical output. For the purposes of this disclosure, an “optical output” is an optical signal that is output from an optical waveguide. As a non-limiting example, the optical waveguide may output the optical output to at least a photodetector. As another non-limiting example, the optical waveguide may output the optical output to a fiber-optic cable. In some embodiments, a design and optimization of the optical waveguides may depend on the wavelength of the optical signal, polarization state, refractive index of materials used and/or mode profile of an output source.

With continued reference to FIGS. 1A-B, a photonic sensor 132 may include one or more resonators. For the purposes of this disclosure, a “resonator” is a structure made of waveguide that can trap, store, transmit, process electromagnetic waves. In an embodiment, the one or more resonators may include photonic crystal cavities, grating structures, or interferometric structures such as Mach-Zehnder or Michelson interferometers, and the like. For the purposes of this disclosure, a “grating structure” is a structure of any regularly spaced collection of essentially identical, parallel, elongated elements, such as but not limited to optical waveguides. A “period A” of the grating determines the diffraction. As a non-limiting example, the grating structures may include a silicon sub-wavelength grating (SWG). For the purposes of this disclosure, a “sub-wavelength grating” is grating structures with a period A that is sufficiently small compared to the wavelength of light.

With continued reference to FIGS. 1A-B, in some embodiments, one or more resonators may include one or more ring resonators. For the purposes of this disclosure, a “ring resonator” is a waveguide that is a closed loop. In some embodiments, the one or more resonators may include various sizes and shapes of the loop (or a ring) and refractive index. In some embodiments, one or more ring resonators may be coupled with an optical waveguide. As a non-limiting example, one or more ring resonators may be in contact with the optical waveguide. As another non-limiting example, the one or more ring resonators may include a gap between the one or more ring resonators and the optical waveguide. In some embodiments, one or more ring resonators may use the principle of resonant wave coupling to filter or select certain wavelengths of light. In some embodiments, the photonic sensor 132 may include one or more arrays of one or more ring resonators. In an embodiment, each of the one or more ring resonators may detect the same one or more analytes of at least a fluid. In another embodiment, each of the one or more ring resonators may detect different one or more analytes of the at least a fluid. As a non-limiting example, one ring resonator of the one or more ring resonators may detect SARS-COV-protein while another ring resonator of the one or more ring resonators detects glucose. When light is input into the loop (or the ring) of one or more ring resonators, the light may circulate around the loop multiple times due to total internal reflection, creating a standing wave pattern with constructive and/or destructive interference. Then, the one or more resonators may output an optical output. Because only a select few wavelengths are at resonance within the loop of the one or more ring resonators, the one or more ring resonators may function as a filter. In an embodiment, the light (or an input optical signal) may be input to a of an optical waveguide of an optical waveguide, where the light may be from a fiber-optic cable with a PM fiber delivering the light from at least a light source. In another embodiment, an optical output may be output from a of an optical waveguide of the optical waveguide to at least a photodetector. As a non-limiting example, the optical output may be output from the of an optical waveguide of the optical waveguide to the at least a photodetector, then, to a reader device 140.

With continued reference to FIGS. 1A-B, in some embodiments, one or more ring resonators may include a single-ring resonator, double-ring resonator, add-drop filter, Vernier ring resonator, Bragg grating ring resonator, and the like. In some embodiments, one or more ring resonators may include a microring resonator. For the purposes of this disclosure, a “microring resonator” is a miniaturized version of the ring resonator. In some embodiments, the microring resonator may be fabricated with a silicon or silicon-on-insulator (SOI) substrate using photolithography, etching, deposition, and/or other microfabrication techniques. For the purposes of this disclosure, “silicon-on-insulator substrate” is a type of semiconductor substrate. As a non-limiting example, the SOI substrate may include a thin layer of silicon, such as but not limited to silicon dioxide, on top of a layer of insulating material which is itself on top of a bulk silicon substrate. In some embodiments, the SOI substrate may reduce capacitance and parasitic effects, provide better isolation between devices, improve radiation hardness, and the like. In some embodiments, the SOI substrate may be fabricated for optical waveguides, ring resonators, and other photonic structures.

With continued reference to FIGS. 1A-B, in some embodiments, one or more resonators may include a respective layer of binding ligands. For the purposes of this disclosure, a “binding ligand” is a ligand that is capable of binding an analyte. Additional disclosure related to the binding ligand disclosed herein may be found in International Patent Application No. PCT/US2022/037767, filed on Jul. 20, 2022, entitled as “WEARABLE BIOSENSORS FOR SEMI-INVASIVE, REAL-TIME MONITORING OF ANALYTES, AND RELATED METHODS AND APPARATUS,” the entirety of which is incorporated herein by reference.

With continued reference to FIGS. 1A-B, in some embodiments, a photonic sensor 132 may utilize evanescent field of an optical waveguide and one or more resonators to probe properties and/or characteristics of the surrounding medium such as but not limited to one or more analytes of at least a fluid. For the purposes of this disclosure, “evanescent field” is a type of electromagnetic field that exists outside the core of an optical waveguide. The evanescent field may decay exponentially with distance from the core and may carry less energy than the propagating mode inside the waveguide. When the waveguide and/or the one or more resonators is brought close to the one or more analytes of the at least a fluid, where the one or more analytes are immobilized on the surface of the waveguide and/or the one or more resonators such as but not limited to with binding ligands, one or more characteristics of the one or more analytes such as but not limited to their concentration, binding kinetics, conformational changes, or the like, may be probed using the evanescent field. Additional disclosure related to various methods to sense the one or more analytes may be found in International Patent Application No PCT/US2022/037767, filed on Jul. 20, 2022, entitled as “WEARABLE BIOSENSORS FOR SEMI-INVASIVE, REAL-TIME MONITORING OF ANALYTES, AND RELATED METHODS AND APPARATUS,” the entirety of which is incorporated herein by reference.

With continued reference to FIGS. 1A-B, in some embodiments, a photonic sensor 132 may include at least a photodetector. In some cases, the photonic sensor 132 may include a plurality of photodetectors, for instance a first photodetector and a second photodetector. In some cases, the first photodetector and/or the second photodetector may be configured to measure one or more of first optical output and second optical output, from a first waveguide and a second waveguide, respectively, such as but not limited to an of an optical waveguide. The at least a first photodetector may be configured to convert the first optical output into a first sensor signal as a function of variance of an optical property of the first waveguide, where the first sensor signal may include without limitation any voltage and/or current waveform. Additionally, or alternatively, the photonic sensor 132 may include a second photodetector located down beam from the second waveguide. In some embodiments, the second photodetector may be configured to measure a variance of an optical property of second waveguide and convert the second optical output into a second sensor signal as a function of the variance of the optical property of the second waveguide.

With continued reference to FIGS. 1A-B, as used in this disclosure, a “photodetector” is any device that is sensitive to light and thereby able to detect light. In some cases, the at least a photodetector may include a photodiode, a photoresistor, a photosensor, a photovoltaic chip, and the like. In some cases, the at least a photodetector may include a Germanium-based photodiode. The at least a photodetector may include, without limitation, Avalanche Photodiodes (APDs), Single Photon Avalanche Diodes (SPADs), Silicon Photomultipliers (SiPMs), Photo-Multiplier Tubes (PMTs), Micro-Channel Plates (MCPs), Micro-Channel Plate Photomultiplier Tubes (MCP-PMTs), Indium gallium arsenide semiconductors (InGaAs), photodiodes, and/or photosensitive or photon-detecting circuit elements, semiconductors and/or transducers. “Avalanche Photo Diodes (APDs),” as used herein, are diodes (e.g., without limitation p-n, p-i-n, and others) reverse biased such that a single photon generated carrier can trigger a short, temporary “avalanche” of photocurrent on the order of milliamps or more caused by electrons being accelerated through a high field region of the diode and impact ionizing covalent bonds in the bulk material, these in turn triggering greater impact ionization of electron-hole pairs. APDs may provide a built-in stage of gain through avalanche multiplication. When the reverse bias is less than the breakdown voltage, the gain of the APD may be approximately linear. For silicon APDs, this gain may be on the order of 10-100. Material of APD may contribute to gains. Germanium APDs may detect infrared out to a wavelength of 1.7 micrometers. InGaAs may detect infrared out to a wavelength of 1.6 micrometers. Mercury Cadmium Telluride (HgCdTe) may detect infrared out to a wavelength of 14 micrometers. An APD reverse biased significantly above the breakdown voltage may be referred to as a Single Photon Avalanche Diode, or SPAD. In this case, the n-p electric field may be sufficiently high to sustain an avalanche of current with a single photon, hence referred to as “Geiger mode.” This avalanche current rises rapidly (sub-nanosecond), such that detection of the avalanche current can be used to approximate the arrival time of the incident photon. The SPAD may be pulled below breakdown voltage once triggered in order to reset or quench the avalanche current before another photon may be detected, as while the avalanche current is active carriers from additional photons may have a negligible effect on the current in the diode.

With continued reference to FIGS. 1A-B, in some cases, at least a photodetector may include a photosensor array, for example without limitation a one-dimensional array. The photosensor array may be configured to detect a variance in an optical property of waveguide. In some cases, first photodetector and/or second photodetector may be wavelength dependent. For instance, and without limitation, first photodetector and/or second photodetector may have a narrow range of wavelengths to which each of first photodetector and second photodetector are sensitive. As a further non-limiting example, each of first photodetector and second photodetector may be preceded by wavelength-specific optical filters such as bandpass filters and/or filter sets, or the like; in any case, a splitter may divide output from optical matrix multiplier as described below and provide it to each of first photodetector and second photodetector. Alternatively, or additionally, one or more optical elements may divide output from waveguide prior to provision to each of first photodetector and second photodetector, such that each of first photodetector and second photodetector receives a distinct wavelength and/or set of wavelengths. For example, and without limitation, in some cases a wavelength demultiplexer may be disposed between waveguides and first photodetector and/or second photodetector; and the wavelength demultiplexer may be configured to separate one or more lights or light arrays dependent upon wavelength. As used in this disclosure, a “wavelength demultiplexer” is a device that is configured to separate two or more wavelengths of light from a shared optical path. In some cases, a wavelength demultiplexer may include at least a dichroic beam splitter. In some cases, a wavelength demultiplexer may include any hot mirror, a cold mirror, a short-pass filter, a long pass filter, a notch filter, and the like. An exemplary wavelength demultiplexer may include part No. WDM-11P from OZ Optics of Ottawa, Ontario, Canada. Further examples of demultiplexers may include, without limitation, gratings, prisms, and/or any other devices and/or components for separating light by wavelengths that may occur to persons skilled in the art upon reviewing the entirety of this disclosure. In some cases, at least a photodetector may be communicative with computing device, such that a sensed signal such as but not limited to one or more sensor signals may be communicated with computing device of a reader device 140.

With continued reference to FIGS. 1A-B, an apparatus 100 includes a reader device 140. For the purposes of this disclosure, a “reader device” is a device that processes signals for, generated by, or received by a photonic sensor. As a non-limiting example, the reader device 140 is configured to detect one or more characteristics of extracted fluid as a function of a sensor signal. For the purposes of this disclosure, a “characteristic” of a fluid is a distinguishing feature of a fluid. As a non-limiting example, the one or more characteristics may include presences of one or more analytes in the at least a fluid, concentration level of the one or more analytes in the at least a fluid, binding kinetics, conformational changes, and the like. Additional disclosure related to the one or more characteristics of the fluid and/or the methods to determine the one or more characteristics of the fluid may be found in International Patent Application No PCT/US2022/037767, filed on Jul. 20, 2022, entitled as “WEARABLE BIOSENSORS FOR SEMI-INVASIVE, REAL-TIME MONITORING OF ANALYTES, AND RELATED METHODS AND APPARATUS,” the entirety of which is incorporated herein as a reference. The reader device 140 may be communicatively connected to the photonic sensor 132. In some embodiments, the reader device 140 may be connected with the photonic sensor 132 using a connecting system as described below. For the purposes of this disclosure, “communicatively connected” means connected by way of a connection, attachment, or linkage between two or more relata which allows for reception and/or transmittance of information therebetween. For example, and without limitation, this connection may be wired or wireless, direct or indirect, and between two or more components, circuits, devices, systems, and the like, which allows for reception and/or transmittance of data and/or signal(s) therebetween. Data and/or signals therebetween may include, without limitation, electrical, electromagnetic, magnetic, video, audio, radio, and microwave data and/or signals, combinations thereof, and the like, among others. A communicative connection may be achieved, for example and without limitation, through wired or wireless electronic, digital or analog, communication, either directly or by way of one or more intervening devices or components. Further, communicative connection may include electrically coupling or connecting at least an output of one device, component, or circuit to at least an input of another device, component, or circuit. For example, and without limitation, via a bus or other facility for intercommunication between elements of a computing device. Communicative connecting may also include indirect connections via, for example and without limitation, wireless connection, radio communication, low power wide area network, optical communication, magnetic, capacitive, or optical coupling, and the like. In some instances, the terminology “communicatively coupled” may be used in place of communicatively connected in this disclosure.

With continued reference to FIGS. 1A-B, a reader device 140 may include a computing device. In some embodiments, a processor and a memory communicatively connected to the processor may be included in the computing device. The computing device may include any computing device as described in this disclosure, including without limitation a microcontroller, microprocessor, digital signal processor (DSP) and/or system on a chip (SoC) as described in this disclosure. The computing device may include, be included in, and/or communicate with a mobile device such as a mobile telephone or smartphone. The computing device may include a single computing device operating independently, or may include two or more computing device operating in concert, in parallel, sequentially or the like; two or more computing devices may be included together in a single computing device or in two or more computing devices. The computing device may interface or communicate with one or more additional devices as described below in further detail via a network interface device. Network interface device may be utilized for connecting the computing device to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Computing device may include but is not limited to, for example, a computing device or cluster of computing devices in a first location and a second computing device or cluster of computing devices in a second location. The computing device may include one or more computing devices dedicated to data storage, security, distribution of traffic for load balancing, and the like. the computing device may distribute one or more computing tasks as described below across a plurality of computing devices of computing device, which may operate in parallel, in series, redundantly, or in any other manner used for distribution of tasks or memory between computing devices. the computing device may be implemented, as a non-limiting example, using a “shared nothing” architecture.

With continued reference to FIGS. 1A-B, a computing device may be designed and/or configured to perform any method, method step, or sequence of method steps in any embodiment described in this disclosure, in any order and with any degree of repetition. For instance, the computing device may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. The computing device may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

With continued reference to FIGS. 1A-B, in some embodiments, a reader device 140 may include one or more elements of dedicated signal processing hardware and/or software modules. This may include filters, filter banks including analysis and synthesis banks, fast Fourier Transform (FFT) calculation modules, signal generators, matrix operation calculators, or the like. In some embodiments, the reader device 140 may be configured to perform one or more signal processing steps on a signal, where the signal is any signal disclosed in the entirety of this disclosure. As used in this disclosure, a “signal” is any intelligible representation of data, for example from one device to another. A signal may include an optical signal, a hydraulic signal, a pneumatic signal, a mechanical signal, an electric signal, a digital signal, an analog signal, and the like. In some cases, a signal may be used to communicate with a computing device, for example by way of one or more ports. In some cases, a signal may be transmitted and/or received by a computing device, for example by way of an input/output port. An analog signal may be digitized, for example by way of an analog to digital converter. In some cases, an analog signal may be processed, for example by way of any analog signal processing steps described in this disclosure, prior to digitization. In some cases, a digital signal may be used to communicate between two or more devices, including without limitation computing devices. In some cases, a digital signal may be communicated by way of one or more communication protocols, including without limitation internet protocol (IP), controller area network (CAN) protocols, serial communication protocols (e.g., universal asynchronous receiver-transmitter [UART]), parallel communication protocols (e.g., IEEE 128 [printer port]), and the like.

With continued reference to FIGS. 1A-B, as a non-limiting example, a reader device 140 may analyze, modify, and/or synthesize a signal representative of characteristic. Exemplary methods of signal processing may include analog, continuous time, discrete, digital, nonlinear, and statistical. Analog signal processing may be performed on non-digitized or analog signals. Exemplary analog processes may include passive filters, active filters, additive mixers, integrators, delay lines, compandors, multipliers, voltage-controlled filters, voltage-controlled oscillators, and phase-locked loops. Continuous-time signal processing may be used, in some cases, to process signals which may vary continuously within a domain, for instance time. Exemplary non-limiting continuous time processes may include time domain processing, frequency domain processing (Fourier transform), and complex frequency domain processing. Discrete time signal processing may be used when a signal is sampled non-continuously or at discrete time intervals (i.e., quantized in time). Analog discrete-time signal processing may process a signal using the following exemplary circuits sample and hold circuits, analog time-division multiplexers, analog delay lines and analog feedback shift registers. Digital signal processing may be used to process digitized discrete-time sampled signals. Commonly, digital signal processing may be performed by a computing device or other specialized digital circuits, such as without limitation an application specific integrated circuit (ASIC), a field-programmable gate array (FPGA), or a specialized digital signal processor (DSP). Digital signal processing may be used to perform any combination of typical arithmetical operations, including fixed-point and floating-point, real-valued and complex-valued, multiplication and addition. Digital signal processing may additionally operate circular buffers and lookup tables. Further non-limiting examples of algorithms that may be performed according to digital signal processing techniques include fast Fourier transform (FFT), finite impulse response (FIR) filter, infinite impulse response (IIR) filter, and adaptive filters such as the Wiener and Kalman filters. In some embodiments, a filter bank may be used, such as but not limited to analysis banks, synthesis banks, FFT filter banks, and the like. Statistical signal processing may be used to process a signal as a random function (i.e., a stochastic process), utilizing statistical properties. For instance, in some embodiments, a signal may be modeled with a probability distribution indicating noise, which then may be used to reduce noise in a processed signal.

With continued reference to FIGS. 1A-B, a reader device 140 may include at least a light source. The reader device 140 may be configured to provide an input optical signal using at least one light source. For the purposes of this disclosure, an “input optical signal” is an optical signal that includes electromagnetic radiation. In some embodiments, the input optical signal may be transmitted over optical fibers. As used in this disclosure, a “light source” is any device configured to emit electromagnetic radiation. As a non-limiting example, electromagnetic radiation may include ultraviolet (UV), visible light, infrared light, and the like. At least a light source may control propagation, direction, polarization, intensity of light waves. In some embodiments, the photonic sensor 132 may include lenses, mirrors, prisms, filters, optical fibers, and the like. In some embodiments, at least a light source may be tuned across the resonances of elements of a photonic sensor 132. In some cases, at least a light source may include a coherent light source, which is configured to emit coherent light, for example a laser. In some cases, at least a light source may include a non-coherent light source configured to emit non-coherent light, for example a light emitting diode (LED). In some cases, at least a light source may emit a light having substantially one wavelength. In some cases, the at least a light source may emit the light having a wavelength range. The light may have a wavelength in an ultraviolet range, a visible range, a near-infrared range, a mid-infrared range, and/or a far-infrared range. For example, in some cases the light may have a wavelength within a range from about 100 nm to about 20 micrometers. In some cases, the light may have a wavelength within a range of about 400 nm to about 2,500 nm. The at least a light source may include, one or more diode lasers, which may be fabricated, without limitation, as an element of an integrated circuit; diode lasers may include, without limitation, a Fabry Perot cavity laser, which may have multiple modes permitting outputting light of multiple wavelengths, a quantum dot and/or quantum well-based Fabry Perot cavity laser, an external cavity laser, a mode-locked laser such as a gain-absorber system, configured to output light of multiple wavelengths, a distributed feedback (DFB) laser, a distributed Bragg reflector (DBR) laser, an optical frequency comb, and/or a vertical cavity surface emitting laser. At least a light source may additionally or alternatively include a light-emitting diode (LED), an organic LED (OLED) and/or any other light emitter. In some cases, at least a light source may be configured to couple light into a photonic sensor 132 for instance into one or more waveguide described above.

With continued reference to FIGS. 1A-B, a reader device 140 may be configured to receive one or more sensor signals from a photonic sensor 132. For the purposes of this disclosure, a “sensor signal” is a signal obtained from a photonic sensor that is related to one or more analytes of at least a fluid. As a non-limiting example, the sensor signal may include an optical signal, electronic signal, and the like. For example and without limitation, the optical signal may include an optical output from a of an optical waveguide of an optical waveguide. For another example and without limitation, the electronic signal may include a signal from at least a photodetector of the photonic sensor 132. As another non-limiting example, one or more sensor signals may include a resonance wavelength shift. For the purposes of this disclosure, a “resonance wavelength shift” is a change in resonant wavelength of a photonic sensor, such as but not limited to a ring resonator, due to one or more analytes of at least a fluid.

With continued reference to FIGS. 1A-B, in some embodiments, a reader device 140 may receive one or more sensor signals from a photonic sensor 132 as the reader device 140 provides an input optical signal to the photonic sensor 132. In an embodiment, the reader device 140 may receive the one or more sensor signals, such as but not limited to an output signal, from an optical waveguide, such as but not limited to a of an optical waveguide of the photonic sensor 132 using an optical fiber. In another embodiment, the reader device 140 may receive one or more sensor signals from at least a photodetector of the photonic sensor 132. As a non-limiting example, the optical fiber and/or the at least a photodetector may receive the output signal using a vertical coupling, edge coupling, a grating coupler, or any couplers thereof. Additionally, vertical coupling, edge coupling, the grating coupler may be implemented in any other components of the photonic sensor 132 and the reader device 140.

With continued reference to FIGS. 1A-B, a reader device 140 is configured to determine one or more characteristics of one or more analytes of at least a fluid as a function of one or more sensor signals. In some embodiments, the reader device 140 may determine one or more characteristics of the one or more analytes of the at least a fluid as the reader device 140 received one or more sensor signals from the photonic sensor 132. In some embodiments, the reader device 140 may include at least a photodetector. At least a photodetector of the reader device 140 disclosed herein may be consistent with any photodetectors described in the entirety of this disclosure. As a non-limiting example, when the reader device 140 receives an optical output, the at least a photodetector of the reader device 140 may receive the optical output and convert the optical output to one or more sensor signals to determine the one or more characteristics of the one or more analytes of the at least a fluid. As a non-limiting example, the one or more characteristics of the one or more analytes of the at least a fluid may include presences of one or more analytes in the at least a fluid, concentration level of the one or more analytes in the at least a fluid, and the like. The one or more characteristics of the one or more analytes may be determined as a function of a change in a resonance wavelength, change in optical wavelength, change of concentration level, and the like. For example and without limitation, a shift in the resonance wavelength of one or more resonators may indicate the presence of the one or more analytes of interest. Additional disclosure related to the one or more characteristics of the one or more analytes and/or the methods to determine the one or more characteristics of the one or more analytes may be found in International Patent Application No PCT/US2022/037767, filed on Jul. 20, 2022, entitled as “WEARABLE BIOSENSORS FOR SEMI-INVASIVE, REAL-TIME MONITORING OF ANALYTES, AND RELATED METHODS AND APPARATUS,” the entirety of which is incorporated herein as a reference.

With continued reference to the FIGS. 1A-B, an apparatus 100 includes an assay component 116. For the purposes of this disclosure, an “assay component” is a component used in the analysis of fluids. In some embodiments, the assay component 116 is fluidically connected to a microfluidic assembly 140 as described above. As a non-limiting example, the assay component 116 is configured to test the extracted fluid to detect one or more characteristics of the extracted fluid. In some embodiments, the assay component 116 may include a lateral flow assay. For the purposes of this disclosure, a “lateral flow assay” is a type of diagnostic test that detects the presence or absence of a target substance in a sample that migrates parallel to a surface on an assay component. In some embodiments, the lateral flow assay may include a paper-based strip. As a non-limiting example, extracted fluid can be applied to one end of the paper-based strip and then migrates along the paper-based strip by capillary action. As the extracted fluid flows through the paper-based strip, it may pass through different zones that include capture molecules that are specific to the target substance (e.g. analyte). For example, and without limitation, the capture molecules may include antibodies, DNA probes, binding ligands, and the like. If the target substance is present in the sample, the target substance may bind to the capture molecules and create a visible signal, such as a colored line, in the detection zone of the paper-based strip. In some embodiments, the assay component 116 may be used for point-of-care testing. For the purposes of this disclosure, “point-of-care diagnostic” is a technique of diagnosis that allows detection and diagnosis of diseases at or near the patient site. For the purposes of this disclosure, a “patient site” is a patient's physical location at the time of receipt of a sample from the patient's body. In some embodiments, the assay component 116 may be configured for a multiplexed diagnostic. For the purposes of this disclosure, “multiplexed diagnostic” is a technique of diagnosis that can detect multiple analytes in a single sample.

With continued reference to the FIGS. 1A-B, in some embodiments, a photonic sensor 132 may be further configured to detect a change in an assay component 116. As a non-limiting example, the photonic sensor 132 may detect a change in a lateral flow assay by using light to measure the intensity of the color that develops in the test line (a color line of the lateral flow assay). When extracted fluid is applied to a paper-based strip, the extracted fluid may flow through the test line where it interacts with specific reagents. As a color change occurs in the test line due to the binding of the analyte and the reagents, the photonic sensor 132 may emit light at a certain wavelength and may measure the intensity of the light that is transmitted through the test line. If a color change has occurred, the intensity of the transmitted light will be different, indicating the presence of the target analyte. This change in light intensity can be detected and processed by the photonic sensor 132 to generate a sensor signal to output to a reader device 140. Then the reader device 140 may analyze the sensor signal to determine characteristics of the extracted fluid as a function of the sensor signal.

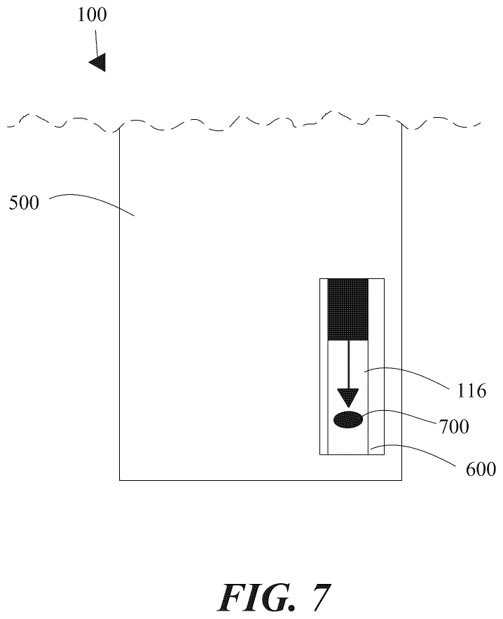

With continued reference to the FIGS. 1A-B, in some embodiments, an assay component 116 may include a test timing feature. For the purposes of this disclosure, a “test timing feature” is a feature of an assay component that indicates when the test is complete or when it is time to read the results. As a non-limiting example, the test timing feature may include an indicator that indicates to a user that the test is complete or it is time to read the results. As another non-limiting example, the indicator may indicate to the user that the assay component 116 can be removed from a housing. As another non-limiting example, the test timing feature may include an indicator that indicates a photonic sensor 132 that the test is complete. An exemplary configuration of the test timing feature of the assay component is shown in FIG. 7.

With continued reference to the FIGS. 1A-B, an apparatus 100 includes a fluid collecting reservoir 124. For the purposes of this disclosure, a “fluid collecting reservoir” is a component that is configured to collect a fluid. In some embodiments, the fluid collecting reservoir 124 is fluidically connected to the microfluidic assembly. As a non-limiting example, the fluid collecting reservoir 124 is configured to collect extracted fluid from a microfluidic assembly 140. As another non-limiting example, the fluid collecting reservoir 124 may be further configured to passively pump the extracted fluid for the flow of the extracted fluid of the microfluidic assembly 140 as described above. In some embodiments, the fluid collecting reservoir 124 may include a fluid collection pad. For the purposes of this disclosure, a “fluid collection pad” is a material or device designed to collect and absorb fluids. In some embodiments, the fluid collection pad may include materials that absorb the fluids. As a non-limiting example, the fluid collection pad may include cotton, cellulose, or other absorbent polymers. In some embodiments, the fluid collecting reservoir 124 may include a fluid collecting container. As another non-limiting example, the fluid collecting reservoir 124 may include a microtiter tube, a dried blood spot, a plasma separating paper, and the like. In some embodiments, the fluid collecting container may include materials that are compatible with the fluids. As a non-limiting example, the fluid collecting container may include plastic, glass, and the like. In some embodiments, the fluid collecting container may include anticoagulants or preservatives to prevent the blood sample from clotting or deteriorating during storage and transportation. In some embodiments, the fluid collecting reservoir 124 may be removably inserted into a housing as described above. Once, in a non-limiting example, the fluid collecting reservoir 124 is removed from the housing, the fluid collecting reservoir 124 can be sent using a mailing system for further testing. In some embodiments, the fluid collecting reservoir 124 may be disposable. In some embodiments, the fluid collecting reservoir 124 may be replaceable. In some embodiments, the fluid collecting reservoir 124 may be used for laboratory based diagnostic. As a non-limiting example, the fluid collecting reservoir 124 may be sent to a laboratory for diagnostic purposes. For example, and without limitation, the fluid collecting reservoir 124 may be mailed to the laboratory and analyzed in the laboratory by a trained personnel. In some embodiments, inside the fluid collecting reservoir 124, the collected fluid may be separated into different streams going to the different immediate test areas and the different storage techniques. In some embodiments, the collected fluid may be either diluted, undiluted or lysed.

Referring now to FIG. 2, an exemplary workflow of an apparatus 100 of a multipurpose fluid extraction device for diagnostics is illustrated. In some embodiments, a photonic sensor 132 may output a sensor signal to the reader device 140. The sensor signal may be enabled by ring resonators and a functionalized surface of the photonic sensor 132 that can facilitate specific binding. For an amplified binding, amplifiers can be placed on microfluidic channels 200 of a microfluidic assembly 140 to later be driven alongside extracted fluid onto the photonic sensor 132. The amplified sensor signal may be reported on/with the reader device 140.

With continued reference to FIG. 2, in some embodiments, a photonic sensor 132 may be communicatively connected to a reader device 140 using a connecting cable. As a non-limiting example, the connecting cable may include a fiber-optic cable, ribbon cable, and the like. For the purposes of this disclosure, a “fiber-optic cable” is a cable that includes one or more optical fibers (also called “optic fiber” or “fiber”) that are used to transmit optical signals. In some embodiments, the fiber-optic cable may transmit any signal disclosed in the entirety of this disclosure, such as but not limited to an input optical signal, output optical signal, sensor signal, and the like. In some embodiments, the fiber-optic cable may be implemented with any elements of a photonic sensor 132 and/or a reader device 140 disclosed in the entirety of this disclosure such as but not limited to an optical waveguide, photodetector, and the like. For the purposes of this disclosure, a “ribbon cable” is a fiber-optic cable with a plurality of optical fibers running parallel to each other on the same flat plane. In some embodiments, the ribbon cable may connect a multi-fiber push on connector (MPO) and the photonic sensor 132 to connect the photonic sensor 132 and the reader device 140. In some embodiments, the fiber-optic cable may include single-mode fibers (SMF), multimode fibers (MF), and the like. Additional disclosure related to the connecting cable may be found in U.S. patent application Ser. No. 18/126,014.

With continued reference to FIG. 2, in some embodiments, an apparatus 100 may be placed on a patient's skin enabling a fluid to flow into the apparatus 100. The flow of extracted fluid may be driven into a microfluidic assembly and led over a photonic sensor 132. The extracted fluid can later be driven to a fluid collecting reservoir 124 and/or an assay component 116. In some embodiments, the patient can later use the collected fluid in the fluid collecting reservoir 124 for further testing. In some embodiments, additionally or alternatively, the patient can also see initial testing on the assay component 116 and/or a reader device 140.

Referring now to FIGS. 3A-C, exemplary embodiments of a fluid extraction device 104 and its use is illustrated. In some embodiments, the fluid extraction device 104 starts fluid extraction by placing and/or attaching the fluid extraction device 104 over the skin 300 of a patient. Then, as a non-limiting example, a mechanical skin piercing component may pierce the skin 300. For example, and without limitation, the mechanical skin piercing component microneedles 304, micro blades 308, and the like as shown in FIGS. 3A-B. In some embodiments, the microneedles 304 and/or micro blades 308 may be actuated using a computing device. In some embodiments, the microneedles 304 and/or micro blades 308 may be actuated manually by the patient using an actuator. As a non-limiting example, the patient may push a button 312 to actuate the microneedles 304 and/or micro blades 308 to pierce the skin 300. As another non-limiting example, an optical skin piercing component may pierce the skin 300. For example, and without limitation, the optical skin piercing component may include a laser lancet 316 as shown in FIG. 3C. In some embodiments, the laser lancet 316 may be actuated using the computing device. In some embodiments, the laser lancet 316 may be actuated manually by the patient using the actuator. As a non-limiting example, the patient may push a button 312 to actuate the laser lancet 316 to pierce the skin 300. By piercing the skin using the fluid extraction system 104, the extracted fluid may flow into a microfluidic assembly 140.

Referring now to FIGS. 4A-D, cross section views of exemplary embodiments of a microfluidic assembly 140 are illustrated. In some embodiments, extracted fluid that is extracted using a fluid extraction system 104 may be driven in a microfluidic assembly 140. In some embodiments, the microfluidic assembly 140 may be formed by etched channels 200 (e.g. microfluidic channels 200) on a substrate described above. In some embodiments, the microfluidic assembly 140 may be formed by etched channels 200 on an injection molded piece 400. For the purposes of this disclosure, an “injection molded piece” is a component or part that has been created through the process of injection molding. For the purposes of this disclosure, “injection molding” is a manufacturing process used to produce parts by injecting molten material into a mold, where it cools and solidifies to form the desired shape. As a non-limiting example, the injection molded piece 400 may include plastics, metals, ceramics, and glass. In some embodiments, the microfluidic assembly 140 may be formed by means of etched PSA 404. For the purposes of this disclosure, a “pressure sensitive adhesive,” also called “PSA” is a type of adhesive that forms a bond when pressure is applied to it. As a non-limiting example, the PSA can be used to create multiple layers of the microfluidic channel 200 by laminating the adhesive over etched elements or other PSA laminates. In some embodiments, the microfluidic assembly 140 may be sealed with adhesive laminates 408. For the purposes of this disclosure, “adhesive laminates” are materials consisting of two or more layers that are bonded together using an adhesive. In some embodiments, the adhesive laminates 408 can be produced using a variety of techniques, such as but not limited to hot melt, solvent-based, or water-based adhesives. In some embodiments, the size of the microfluidic channels 200 may depend on capillary needs as well as fluid characteristics.

Referring now to FIGS. 5A-B, exemplary embodiments of a portion of an apparatus 100 with an assay component 116 and a fluid collecting reservoir 124 removably connected to a housing 500 are illustrated. In an embodiment, the assay component 116 may be removably inserted in the housing 500 through a first aperture 120 of the housing 500. For example, and without limitation, the assay component 116 may be removably inserted into the housing 500 through the first aperture 120 for testing at least a fluid and removed once the testing is finished. As another non-limiting example, the fluid collecting reservoir 124 may be removably inserted in the housing 500 through a second aperture 128 as shown in FIGS. 5A-B. For example, and without limitation, the fluid collecting reservoir 124 may be removably inserted into the housing 500 through the second aperture 128 for collecting the at least a fluid and removed through the second aperture 128 once the at least a fluid is collected. In some embodiments, the fluid collecting reservoir 124 can serve a dual purpose of capillary pump and blood collector as described with respect to FIGS. 1A-B. As extracted fluid reaches the fluid collecting reservoir 124, the extracted fluid may wick into the fluid collecting reservoir 124, driving flow in the microfluidic channels 200 of a microfluidic assembly 140. In some embodiments, distance from fluid origin may depend on wicking capabilities and microfluidic elements resistance. After the run is complete. In some embodiments, fluid collecting reservoir 124 may be collected to be sent for further testing. Alternatively, the whole device (apparatus 100) may be sent for further testing.

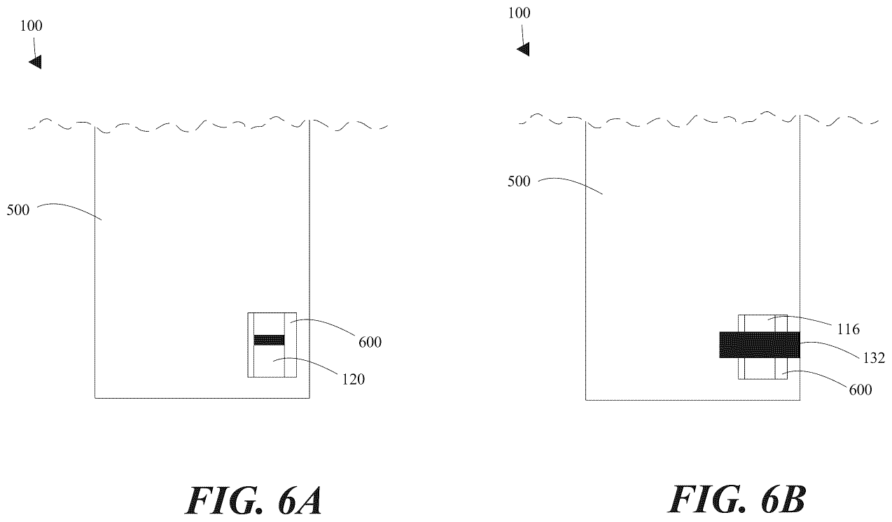

Referring now to FIGS. 6A-B, exemplary embodiments of a portion of an apparatus 100 with an assay component 116 and a photonic sensor 132 are illustrated. In some embodiments, the assay component 116 may serve a dual purpose of a lateral flow assay and capillary pump. As fluid reaches the assay component 116, in a non-limiting example, plasma may get separated and the lateral flow assay may be conducted. In some embodiments, a housing 500 may include a fifth aperture 600, where a process of testing and the result using the assay component 116 can be shown through the fifth aperture 600. In some embodiments, the photonic sensor 132 may be removably connected to the housing 500 on atop of the assay component 116 as shown in FIG. 6B. In some embodiments, a photonic sensor 132 may be further configured to detect a change in an assay component 116. As a non-limiting example, the photonic sensor 132 may detect a change in a lateral flow assay by using light to measure the intensity of the color that develops in the test line (a color line of the lateral flow assay). As a non-limiting example, the testing result of the assay component 116 may be reported as a colorimetric change perceived by a patient. As another non-limiting example, the testing result of the assay component 116 may be reported by means of the photonic sensor 132.

Referring now to FIG. 7, an exemplary embodiment of a portion of an apparatus 100 with an assay component 116 and a test timing feature 700 is illustrated. In some embodiments, the assay component 116 may include a test timing feature 700. As a non-limiting example, the test timing feature may include an indicator to show microfluidic flow or/and when to remove the apparatus 100 from a patient. As a non-limiting example, the test timing feature 700 may include an indicator that indicates to the patient that the test is complete or it is time to read the results. As another non-limiting example, the indicator may indicate to the patient that the assay component 116 can be removed from a housing 500. As another non-limiting example, the test timing feature 700 may include an indicator that indicates a photonic sensor 132 that the test is complete.

In general, the cartridge has multiple purposes. It can either draw blood or take in a blood or other sample. Nevertheless, having the cartridge and blood drawing device integrated into the same physical object greatly enhances user experience and makes it such that the user will not need to handle complex matrix media, e.g. blood. The user will also not see any blood, making it more accessible to the greater audience and people with hemophobia. This could also be accomplished by a blood drawing device that is then inserted into the cartridge (without blood being shown), at the cost of more complexity. This would also decrease the duration of the cartridge reagents coming in contact with the atmosphere.

The description herein is provided with the intention to enable a person having ordinary skill in the art to make use of the disclosure. The description herein, with connection to the drawings, describes example configurations and does not represent all the examples that may be implemented or that are within the scope of the present disclosure. Various modifications to the details provided within the disclosure will be apparent to a person having ordinary skill in the art, and the general principles outlined herein may be applied to other variations without departing from the scope of the disclosure. The term “example” means “serving as an example, illustration, or instance” and not “preferred over other examples.”

The specific details in the detailed description included are provided for the purpose of providing an understanding of the described techniques. These techniques, however, may be practiced with or without these specific details. In some cases, known structures and devices are presented in broad detail to avoid obscuring the concepts of the described examples. Thus, the disclosure is not limited to the examples and designs described herein but is to be conferred the broadest scope consistent with the principles and novel features disclosed herein.

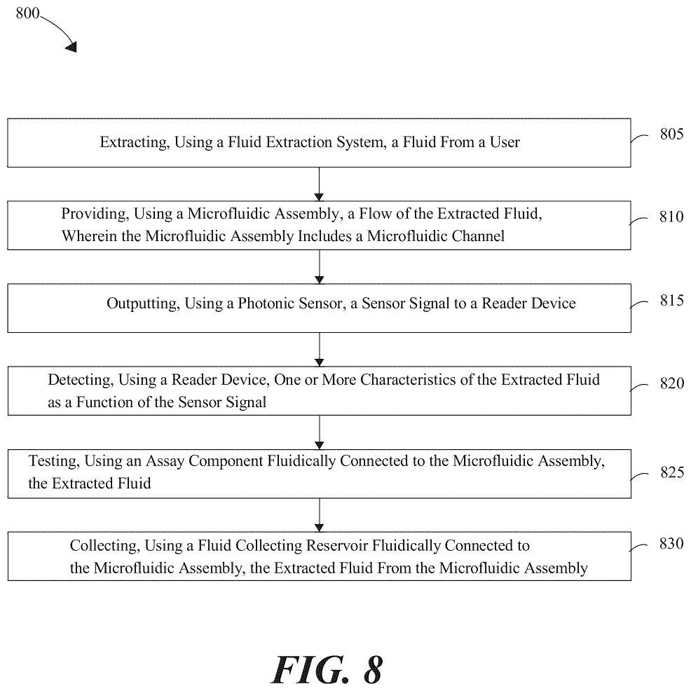

Referring now to FIG. 8, a flow diagram of an exemplary method 800 of use of a multipurpose fluid extraction device for diagnostics. The method 800 includes a step 805 of extracting, using a fluid extraction system, a fluid from a user. In some embodiments, the apparatus may further include a housing, wherein the housing may be portable. In some embodiments, the fluid extraction system may include an optical skin piercing component. These may be implemented as disclosed with respect to FIG. 1-7.

With continued reference to FIG. 8, a method 800 includes a step 810 of providing, using a microfluidic assembly, a flow of extracted fluid, wherein the microfluidic assembly includes a microfluidic channel. In some embodiments, the method 800 may further include providing, using the microfluidic assembly, the flow of the extracted fluid over the photonic sensor. These may be implemented as disclosed with respect to FIG. 1-7.

With continued reference to FIG. 8, a method 800 includes a step 815 of outputting, using a photonic sensor, a sensor signal to a reader device. In some embodiments, the photonic sensor may include a microring resonator. In some embodiments, the method 800 may further include detecting, using the photonic sensor, a color change of the lateral flow assay of the assay component. These may be implemented as disclosed with respect to FIG. 1-7.

With continued reference to FIG. 8, a method 800 includes a step 820 of detecting, using a reader device, one or more characteristics of the extracted fluid as a function of the sensor signal. These may be implemented as disclosed with respect to FIG. 1-7.

With continued reference to FIG. 8, a method 800 includes a step 825 of testing, using an assay component fluidically connected to the microfluidic assembly, the extracted fluid. In some embodiments, the method 800 may further include removably inserting the assay component into a housing through a first aperture of the housing. In some embodiments, the assay component may include a lateral flow assay. These may be implemented as disclosed with respect to FIG. 1-7.

With continued reference to FIG. 8, a method 800 includes a step 830 of collecting, using a fluid collecting reservoir fluidically connected to the microfluidic assembly, the extracted fluid from the microfluidic assembly. In some embodiments, the method 800 may further include removably inserting the fluid collecting reservoir into a housing through a second aperture of the housing. In some embodiments, the method 800 may further include passively pumping, using the fluid collecting reservoir, the extracted fluid for the flow of the extracted fluid of the microfluidic assembly. These may be implemented as disclosed with respect to FIG. 1-7.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

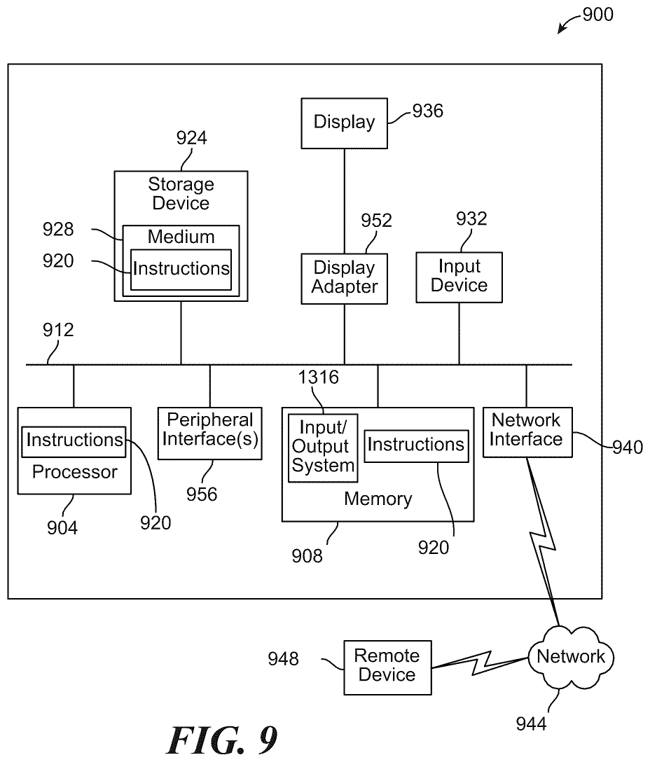

FIG. 9 shows a diagrammatic representation of one embodiment of a computing device in the exemplary form of a computer system 900 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 900 includes a processor 904 and a memory 908 that communicate with each other, and with other components, via a bus 912. Bus 912 may include any of several types of bus structures including, but not limited to, a memory bus, a memory controller, a peripheral bus, a local bus, and any combinations thereof, using any of a variety of bus architectures.

Processor 904 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 904 may be organized according to Von Neumann and/or Harvard architecture as a non-limiting example. Processor 904 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), a System on Module (SOM), and/or system on a chip (SoC).

Memory 908 may include various components (e.g., machine-readable media) including, but not limited to, a random-access memory component, a read only component, and any combinations thereof. In one example, a basic input/output system 916 (BIOS), including basic routines that help to transfer information between elements within computer system 900, such as during start-up, may be stored in memory 908. Memory 908 may also include (e.g., stored on one or more machine-readable media) instructions (e.g., software) 920 embodying any one or more of the aspects and/or methodologies of the present disclosure. In another example, memory 908 may further include any number of program modules including, but not limited to, an operating system, one or more application programs, other program modules, program data, and any combinations thereof.

Computer system 900 may also include a storage device 924. Examples of a storage device (e.g., storage device 924) include, but are not limited to, a hard disk drive, a magnetic disk drive, an optical disc drive in combination with an optical medium, a solid-state memory device, and any combinations thereof. Storage device 924 may be connected to bus 912 by an appropriate interface (not shown). Example interfaces include, but are not limited to, SCSI, advanced technology attachment (ATA), serial ATA, universal serial bus (USB), IEEE 994 (FIREWIRE), and any combinations thereof. In one example, storage device 924 (or one or more components thereof) may be removably interfaced with computer system 900 (e.g., via an external port connector (not shown)). Particularly, storage device 924 and an associated machine-readable medium 928 may provide nonvolatile and/or volatile storage of machine-readable instructions, data structures, program modules, and/or other data for computer system 900. In one example, software 920 may reside, completely or partially, within machine-readable medium 928. In another example, software 920 may reside, completely or partially, within processor 904.

Computer system 900 may also include an input device 932. In one example, a user of computer system 900 may enter commands and/or other information into computer system 900 via input device 932. Examples of an input device 932 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 932 may be interfaced to bus 912 via any of a variety of interfaces (not shown) including, but not limited to, a serial interface, a parallel interface, a game port, a USB interface, a FIREWIRE interface, a direct interface to bus 912, and any combinations thereof. Input device 932 may include a touch screen interface that may be a part of or separate from display 936, discussed further below. Input device 932 may be utilized as a user selection device for selecting one or more graphical representations in a graphical interface as described above.

A user may also input commands and/or other information to computer system 900 via storage device 924 (e.g., a removable disk drive, a flash drive, etc.) and/or network interface device 940. A network interface device, such as network interface device 940, may be utilized for connecting computer system 900 to one or more of a variety of networks, such as network 944, and one or more remote devices 948 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 944, may employ a wired and/or a wireless mode of communication. In general, any network topology may be used. Information (e.g., data, software 920, etc.) may be communicated to and/or from computer system 900 via network interface device 940.

Computer system 900 may further include a video display adapter 952 for communicating a displayable image to a display device, such as display device 936. Examples of a display device include, but are not limited to, a liquid crystal display (LCD), a cathode ray tube (CRT), a plasma display, a light emitting diode (LED) display, and any combinations thereof. Display adapter 952 and display device 936 may be utilized in combination with processor 904 to provide graphical representations of aspects of the present disclosure. In addition to a display device, computer system 900 may include one or more other peripheral output devices including, but not limited to, an audio speaker, a printer, and any combinations thereof. Such peripheral output devices may be connected to bus 912 via a peripheral interface 956. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve apparatuses and methods according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions, and additions may be made to that which is specifically disclosed herein without departing from the spirit and scope of the present invention.

What is claimed is:

1. An apparatus of a multipurpose fluid extraction device for diagnostics, the apparatus comprising:
a fluid extraction system, wherein the fluid extraction system is configured to extract a fluid from a user;
a microfluidic assembly, wherein the microfluidic assembly is configured to provide a flow of the extracted fluid, wherein the microfluidic assembly comprises a microfluidic channel;
a photonic sensor, wherein the photonic sensor is configured to output a sensor signal to a reader device;
a reader device, wherein the reader device is configured to detect one or more characteristics of the extracted fluid as a function of the sensor signal;
an assay component fluidically connected to the microfluidic assembly, wherein the assay component is configured to test the extracted fluid; and
a fluid collecting reservoir fluidically connected to the microfluidic assembly, wherein the fluid collecting reservoir is configured to collect the extracted fluid from the microfluidic assembly.

2. The apparatus of claim 1, further comprising a housing, wherein the housing is portable.

3. The apparatus of claim 2, wherein the assay component is removably inserted into the housing through a first aperture of the housing.

4. The apparatus of claim 2, wherein the fluid collecting reservoir is removably inserted into the housing through a second aperture of the housing.

5. The apparatus of claim 1, wherein the fluid extraction system comprises an optical skin piercing component configured to pierce a skin of the user.

6. The apparatus of claim 1, wherein the microfluidic assembly is further configured to provide the flow of the extracted fluid over the photonic sensor.

7. The apparatus of claim 1, wherein the photonic sensor comprises a microring resonator.

8. The apparatus of claim 1, wherein the assay component comprises a lateral flow assay.

9. The apparatus of claim 8, wherein the photonic sensor is further configured to detect a change in the lateral flow assay of the assay component.

10. The apparatus of claim 1, wherein the fluid collecting reservoir is further configured to passively pump the extracted fluid for the flow of the extracted fluid of the microfluidic assembly.

11. A method of use of a multipurpose fluid extraction device for diagnostics, the method comprising:
extracting, using a fluid extraction system, a fluid from a user;
providing, using a microfluidic assembly, a flow of the extracted fluid, wherein the microfluidic assembly comprises a microfluidic channel;
outputting, using a photonic sensor, a sensor signal to a reader device;
detecting, using a reader device, one or more characteristics of the extracted fluid as a function of the sensor signal;
testing, using an assay component fluidically connected to the microfluidic assembly, the extracted fluid; and
collecting, using a fluid collecting reservoir fluidically connected to the microfluidic assembly, the extracted fluid from the microfluidic assembly.

12. The method of claim 11, further comprising a housing, wherein the housing is portable.

13. The method of claim 12, further comprising:
removably inserting the assay component into the housing through a first aperture of the housing.

14. The method of claim 12, further comprising:
removably inserting the fluid collecting reservoir into the housing through a second aperture of the housing.

15. The method of claim 11, further comprising:
piercing, using an optical skin piercing component of the fluid extraction system, a skin of the user.

16. The method of claim 11, further comprising:
providing, using the microfluidic assembly, the flow of the extracted fluid over the photonic sensor.

17. The method of claim 11, wherein the photonic sensor comprises a microring resonator.

18. The method of claim 11, wherein the assay component comprises a lateral flow assay.

19. The method of claim 11, further comprising:
detecting, using the photonic sensor, a change in the lateral flow assay of the assay component.

20. The method of claim 11, further comprising:
passively pumping, using the fluid collecting reservoir, the extracted fluid for the flow of the extracted fluid of the microfluidic assembly.
